(19) United States
(12) Patent Application Publication
(10) Pub. No.: US 2022/0310271 A1
(43) Pub. Date: Sep. 29, 2022

(54) METHODS FOR THE AUTOMATIC CONSTRUCTION OF STATE TRANSITION GRAPHS FROM THE TIMELINE DATA OF INDIVIDUALS

(71) Applicant: KONINKLIJKE PHILIPS N.V., EINDHOVEN (NL)

(72) Inventors: Yee Him CHEUNG, Boston, MA (US); Alex Ryan MANKOVICH, Somerville, MA (US)

(21) Appl. No.: 17/635,709

(22) PCT Filed: Aug. 21, 2020
(86) PCT No.: PCT/EP2020/073490

§ 371 Date: Feb. 16, 2022

Related U.S. Application Data
(60) Provisional application No. 62/891,593, filed on Aug. 26, 2019.

(51) Int. Cl. G16H 70/20; G16H 10/60; G16H 20/00

(52) U.S. Cl. CPC G16H 70/20; G16H 10/60; G16H 20/00

(57) ABSTRACT

A computer-implemented method for constructing a state transition graph, wherein the method includes obtaining data that includes treatment history and clinical data of a cohort of patients; and generating, by the one or more computing devices, individual treatment pathways for individual patients of the cohort of patients using the treatment history and clinical data for the individual patients; wherein the individual treatment pathways are generated using user-defined parameters including: one or more qualifying events; one or more response states to the one or more qualifying events; and one or more reversible or collapsible events. The method additionally includes constructing a state transition graph that represents multiple aligned and merged individual treatment pathways including the one or more qualifying events, the one or more response states to the one or more qualifying events and the one or more reversible or collapsible events.

### TECHNICAL FIELD

This disclosure relates generally to methods for construction of graphical structures from individual clinical pathways to support predictive modeling of clinical phenotypes or clinical outcomes.

### BACKGROUND

The variation in diagnostic and therapeutic pathways is a well-known deficiency in the current healthcare ecosystem; two physicians treating two patients with identical patient profiles may still prescribe treatments of varying cost or outcome. Currently, there are no known data-driven approaches to personalized care pathway management, partly due to lack of graphical and/or data structures to store and associate historical pathway data and also a lack of appropriate analytical methods to make use of such structures.

Additionally, in genome informatics, a crucial stage of next generation sequencing is the secondary analysis of reads coming from the sequencer. The standard operating procedure for human genomes is that samples are de-multiplexed, aligned to the human reference genome, and algorithmically inspected for aberrations (e.g., variant calling, germline testing, expression analysis, fusion analysis, etc.). All findings subsequent to alignment are dependent on the quality of alignment and the quality of the reference genome itself. The human reference genome, however, is not perfect; it is a single linear sequence based on the consensus of a small number of individuals and does not embody the rich diversity of sequences in the human population. This leads to several practical issues, including misalignment (reads mapped to the wrong position on the genome) or non-alignment (reads not mapped at all), resulting in broad inaccuracies (false positives, false negatives) in clinically relevant and highly variable, regions of the genome. The most promising, albeit relatively nascent, approach to improve the reference is to construct a graph of genomes, where each sample is represented by a path in the graph. A graph-based structure would allow clinicians to capture and discover the diversity of genotypes or haplotypes—and, importantly, complex ones—in the human population, enabling more accurate read alignment.

### SUMMARY

A brief summary of various example embodiments is presented below. Some simplifications and omissions may be made in the following summary, which is intended to highlight and introduce some aspects of the various example embodiments, but not to limit the scope of the invention. Detailed descriptions of example embodiments adequate to allow those of ordinary skill in the art to make and use the inventive concepts will follow in later sections.

Various embodiments relate to a computer-implemented method for constructing a state transition graph for treatment, procedure and progression workflows, wherein the method includes obtaining, by one or more computing devices, data that includes treatment history and clinical data of a cohort of patients; and generating, by the one or more computing devices, individual treatment pathways for individual patients of the cohort of patients using the treatment history and clinical data for the individual patients; wherein the individual treatment pathways are generated using user-defined parameters including: one or more qualifying events; one or more response states to the one or more qualifying events; and one or more reversible or collapsible events. The method additionally includes constructing, by the one or more computing devices, a state transition graph that represents multiple aligned and merged individual treatment pathways including the one or more qualifying events, the one or more response states to the one or more qualifying events and the one or more reversible or collapsible events.

Various embodiments further relate to the one or more qualifying events including one or more treatment regimens, selected from a group that includes a drug regimen, a surgical protocol, a collection of eligible interventions, or combinations thereof.

Various embodiments further relate to the one or more response states being selected from a group that includes a response status after treatment and a subtype of the patient based on a specific gene signature. In various embodiments, the response state may be linked to one or more reports selected from the group consisting of a clinical report, a radiology report, a pathology report, a genomics report, or combinations thereof.

Various embodiments further relate to the constructing step including adding individual treatment pathways one at a time to the state transition graph.

Various embodiments further relate to the state transition graph including edges that correspond to treatments of a similar nature, wherein the edges are collapsible.

Various embodiments further relate to constructing one or more subgraphs generated using further user-defined parameters comprising one or more qualifying events; one or more response states to the one or more qualifying events; and one or more reversible or collapsible events.

Various embodiments relate to a system for processing treatment and clinical data including a storage area to store an algorithm; a processor configured to implement the algorithm to obtain data that includes treatment history and clinical data of a cohort of patients; generate individual treatment pathways for individual patients of the cohort of patients using the treatment history and clinical data for the individual patients using user-defined parameters including: one or more qualifying events; one or more response states to the one or more qualifying events; and one or more reversible or collapsible events; and construct a state transition graph that represents multiple aligned and merged individual treatment pathways including the one or more qualifying events, the one or more response states to the one or more qualifying events and the one or more reversible or collapsible events.

Various embodiments relate to a system for processing treatment and clinical data, wherein the processor is configured to add individual treatment pathways one at a time to the state transition graph.

Various embodiments relate to a system for processing treatment and clinical data, wherein the processor is configured to link the one or more response states to one or more reports selected from the group consisting of a clinical report, a radiology report, a pathology report, a genomics report, or combinations thereof.

Various embodiments also relate to a non-transitory, machine-readable medium storing instructions for controlling a processor to perform operations which include obtaining, by one or more computing devices, data that comprises treatment history and clinical data of a cohort of patients; generating, by the one or more computing devices, individual treatment pathways for individual patients of the cohort of patients using the treatment history and clinical data for the individual patients; wherein the individual treatment pathways are generated using user-defined parameters including one or more qualifying events; one or more response states to the one or more qualifying events; and one or more reversible or collapsible events; and constructing, by the one or more computing devices, a state transition graph that represents multiple aligned and merged individual treatment pathways including the one or more qualifying events, the one or more response states to the one or more qualifying events and the one or more reversible or collapsible events.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate example embodiments of concepts found in the claims and explain various principles and advantages of those embodiments.

These and other more detailed and specific features are more fully disclosed in the following specification, reference being had to the accompanying drawings, in which:

FIG. 1 illustrates an embodiment of a treatment-event state transition graph that summarizes possible treatment paths and corresponding state transitions;

FIG. 2 illustrates an embodiment of a state transition subgraph that summarizes a range of genomic coordinates for genome analysis;

FIG. 3 illustrates an example of the construction of a state transition graph by the alignment and merging of three pathways; and

FIG. 4 illustrates an example of a treatment graph for HCC patients who have met the Milan Criteria for Liver Transplantation.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various example embodiments described herein are not necessarily mutually exclusive, as some example embodiments can be combined with one or more other example embodiments to form new example embodiments. Descriptors such as “first,” “second,” “third,” etc., are not meant to limit the order of elements discussed, are used to distinguish one element from the next, and are generally interchangeable. Values such as maximum or minimum may be predetermined and set to different values based on the application.

State transition graphs may be utilized to effectively aggregate and visualize historical patient data, including disease status, treatment response, and other metadata of a cohort of individual patients, and can support the exploration and discovery of trends and associations between treatments and outcomes through downstream data analysis. This may enable the building of statistical models which make use of that data for aggregated studies, for example, on the effectiveness of certain drugs or treatment courses, or pathway guidance for individual patients optimized for variables such as specific clinical outcomes, cost and minimum side effects.

Example embodiments herein describe systems configured to construct graphical structures optimized for use in predictive modeling of clinical phenotypes or outcomes. Example embodiments further describe methods for constructing the graphical structures that enable graph-based predictive modeling of clinical phenotypes or outcomes. In various embodiments, the graphical structure may include a state transition graph which may be used to summarize treatment/procedural workflows, events of disease progression (e.g., mutational/clonal evolution in a tumor, symptom journey, etc.), combinations of genomic haplotypes (in graph-based genomes), or other information of individual samples. In various embodiments, computational analysis on the graphs together with clinical data, such as disease status and treatment response, of individual samples, may further allow for statistical models to be constructed to infer disease status and progression and clinical outcome, or guide treatment planning by predicting the best clinical pathway for optimizing outcomes.

In various embodiments, the state transition graphs constructed using the method of the disclosure include clinical state transition graphs that summarize treatment data of a cohort of patients through proper alignment and merging of individual treatment pathways. In various embodiments, the state transition graphs may be constructed from timestamped treatment history and clinical data of a cohort of patients through proper alignment and merging of individual treatment pathways. In other embodiments, the state transition graphs may be utilized in other areas such as logistics and operations.

In various embodiments, the state transition graphs may graph a chronology of events. In various embodiments, the chronology of events may include an applied treatment, a logistic procedure, or the emergence of deleterious mutations, and may be represented by directed edges in the graph that cause the transition of an individual from a first response state to a second response state. Additional events, such as cost, drug toxicity, and the like, may be assigned to each edge to allow for ranking of pathways based on cumulative cost, maximum drug toxicity and other relevant measures known to one of skill in the art. In various embodiments, a response state may be used to summarize the transient overall condition. Exemplary response states include therapeutic response and clinical observations and symptoms of individual samples, and may be represented by a vertex in the graph.

In various embodiments, the system may then be configured to generate a combined transition graph for all sampled patients by adding one patient pathway at a time. Patient pathways may be aligned in different ways. In one embodiment, a largest possible sequence of state-event-state units are first identified in a new patient sample that may be matched to the combined graph based on a user-defined similarity criteria and measure. In various embodiments, users may define sets of equivalent response states and events, or rules and conditions for their equivalence. For example, event edges that correspond to treatments of a similar nature may be set as equivalent to simplify the graph and improve the power of detecting clinical associations with more patient samples in an aggregate group. Additionally, users may define multiple specific sequences of events and states, along with their priorities in serving as anchor points for adding a new sample. A sample patient pathway may then be added in such a way that the resultant graph has the least number of additional response states and edges and remains acyclic (no backward transitions). For each individual patient, a response state may be tied to one or a combination of clinical, radiology, pathology and genomics reports as well as electronic health records in that response state.

In various embodiments, the clinical data associated with each response state may enable more sophisticated downstream query or analysis. In various embodiments, the system and method of the disclosure may include an algorithm configured to match a trait under study to a statistical test.

In various embodiments, users may evaluate the potential influence of each edge, e.g., a genomic variant, or a group of edges, e.g., a treatment procedure with different input and output states, on different categorical/quantitative traits, therapeutic responses, clinical outcomes or other computed metrics and labels, by analyzing all samples associated with the edge. In various embodiments users may decide whether to study the effects that emerge immediately after a qualifying event (e.g., the response state reported in the clinical test immediately after a treatment), or during/after a period of time or over the course of a certain number of transitions. In various embodiments, users may also choose to apply a statistical evaluation on a group of edges, e.g., a series of drug administrations, to study their aggregate effects. In various embodiments of the system of the disclosure, based on the nature of the phenotype/outcome variable under study, such as static/dynamic, categorical/quantitative, the system of the disclosure may further be configured to automatically suggest/apply the appropriate statistical tests as described herein.

In some embodiments, the group of edges under study include a static categorical trait. In various embodiments, the static categorical trait may be retrospective, wherein its value for each sample may remain the same along a path, e.g. disease status, and the static categorical trait may have k categorical values category_i. For such static categorical trait, the influence of an edge/path may be evaluated by comparing the distribution of the categories before and after selection by the edge/path. In various embodiments, this may be done by first computing a contingency table (Table 1) that summarizes the number of samples in each category before and after selection (mᵢ, nᵢ).

TABLE 1

| | Trait | | | | |
|---|---|---|---|---|---|
| | Category_1 | Category_2 | . . . | Category_k | Total |
| Selected | n₁ | n₂ | . . . | nₖ | n |
| Not Selected | m₁ − n₁ | m₂ − n₂ | . . . | mₖ − nₖ | m − n |
| Before | m₁ | m₂ | . . . | mₖ | m |
| Selection | | | | | |

Depending on the question to be answered, different metrics and association tests may be computed based on the contingency table for the evaluation of the impact of the edge/path on the static categorical trait.

In various embodiments, suitable metrics and association tests for evaluation of a static categorical trait include a Relative Risk (RR) test, an Odds Ratio (OR), Chi-Square Test of Independence, Fisher's Exact Test of Independence. In various embodiments of the system of the disclosure, the system may be configured to automatically suggest or apply the appropriate metric and association tests for optimal evaluation of the static categorical trait.

In some embodiments, a relative risk (RR) test may be suggested and applied. In this embodiment, for each category i, a relative risk RRᵢ can be computed based on all samples or samples of a designated subset of categories. With reference to contingency Table 1, if it is based on all samples, then

$$R {R}_{i} = \frac{{n}_{i} / n}{( {m}_{i} - {n}_{i} ) / ( m - n )} .$$

If it is based on a designated subset of categories S, then

$$R {R}_{i} = \frac{{n}_{i} / {\sum }_{j ∈ S} {n}_{j}}{( {m}_{i} - {n}_{i} ) / {\sum }_{j ∈ S} ( {m}_{j} - {n}_{j} )} .$$

In some embodiments, an odds ratio (OR) test may be suggested and applied. In this embodiment, for each category i, an odds ratio ORᵢ can be computed based on all samples or samples of a designated subset of categories. With reference to contingency Table 1, if it is based on all samples, then

$${OR}_{i} = \frac{{n}_{i} / ( n - {n}_{i} )}{( {m}_{i} - {n}_{i} ) / [ ( m - n ) - ( {m}_{i} - {n}_{i} ) ]} .$$

If it is based on a designated subset of categories S, then

$$R {R}_{i} = \frac{{n}_{i} / {\sum }_{j ∈ \{ S - i \}} {n}_{j}}{( {m}_{i} - {n}_{i} ) / {\sum }_{j ∈ \{ S - i \}} ( {m}_{j} - {n}_{j} )} .$$

In some embodiments, a Chi-Squre Test of Independence may be suggested and applied. In some embodiments, the Chi-Squre Test of Independence may be applied to test how likely it is that a trait is completely independent from the edge/path. With reference to contingency Table 1, the chi-square statistic is given by

$${𝒳}^{2} = {\sum }_{i = 1}^{k} \frac{{( {n}_{i} - \frac{n \cdot {m}_{i}}{m} )}^{2}}{\frac{n \cdot {m}_{i}}{m}} + {\sum }_{i = 1}^{k} \frac{{[ ( {m}_{i} - {n}_{i} ) - \frac{( m - n ) \cdot {m}_{i}}{m} ) ]}^{2}}{\frac{( m - n ) \cdot {m}_{i}}{m}} .$$

A p value may then be computed based on the Chi-Square distribution with (k−1) degrees of freedom. In various embodiments, the Chi-Square test may best be applied for large sample sizes, e.g., with expected numbers in each category >5. Additionally, in various embodiments having smaller sample sizes, Yates' (for one degree of freedom) or Williams' correction can be automatically applied for improved accuracy.

In some embodiments, a Fisher's Exact Test of Independence may be suggested and applied. In this embodiment, the Fisher's Exact Test of Independence returns exact p values at a higher computational cost, and may best be applied for small sample sizes. In various embodiments, the Fisher's Exact Test of Independence may be generalized for higher dimensional tables, for example, using a Freeman-Halton extension. In other embodiments, the Fisher's Exact Test of Independence may be performed as multiple tests that compare two categories at a time. Depending on the purpose, users may apply the test on all or a subset of the possible category pairs. In various embodiments, comparison can also be made for one category against the rest of the samples pooled together, or between any two groups, with each consisting of a pool of multiple categories. In various embodiments, the overall p value can then be given by the minimum of the p values of individual comparisons, with correction for multiple testing using methods such as Bonferroni and false discovery rate (FDR) adjustments. In one embodiment, the p value for a one-tailed Fisher's Exact Test (k=2) for an increased number of selected samples of Category_1 is given by

$$p = {\sum }_{x = {n}_{1}}^{{m}_{1}} \frac{( \begin{matrix}n \\ x\end{matrix} ) ( \begin{matrix}m - n \\ {m}_{1} - x\end{matrix} )}{( \begin{matrix}m \\ {m}_{1}\end{matrix} )} ,$$

where

$$( \begin{matrix}a \\ b\end{matrix} )$$

is the binomial coefficient.

In various embodiments, the group of edges under study may include a dynamic categorical trait. In various embodiments, the dynamic categorical trait under study may be longitudinal and its value in a sample may change after traversing an edge, e.g., high/low blood pressure, and the trait has k categorical values category_i. For such kind of dynamic categorical traits, the influence of an edge/path may be evaluated by comparing the number of samples that move into and out of each category. This may be done by first computing a contingency table (Table 2) that summarizes the number of samples that remain in category i (mᵢᵢ), or change from category i to category j (mᵢⱼ) after traversing the edge/path.

TABLE 2

| | Trait After | | | | |
|---|---|---|---|---|---|
| | Category_1 | Category_2 | . . . | Category_k | Total |
| Category_1 | m₁₁ | m₁₂ | . . . | m₁ₖ | r₁ |
| Category_2 | m₂₁ | m₂₂ | . . . | m₂ₖ | r₂ |
| . | . | . | . | . | . |
| . | . | . | . | . | . |
| . | . | . | . | . | . |
| Category_k | mₖ₁ | mₖ₂ | . . . | mₖₖ | rₖ |
| Total | c₁ | c₂ | | cₖ | N |

In various embodiments, the contingency table may be configured to show the number of samples remaining in one category (main diagonal) or switching from one category (row) to another (column) after traversing an edge/path. Depending on the question to be answered, different metrics and association tests may be computed based on the contingency table for the evaluation of the impact of the edge/path on the dynamic categorical trait.

In various embodiments, suitable metrics and association tests for evaluation of a dynamic categorical trait include McNemar's Test of Homogeneity of Marginal Distributions and the like. In various embodiments of the system of the disclosure, the system may be configured to automatically suggest or apply the appropriate metric and association tests for optimal evaluation of the dynamic categorical trait.

In various embodiments, the contingency table may be computed by first measuring number/fraction of outgoing samples. In one embodiment, the number/fraction of outgoing samples are computed to measure the number/fraction of samples that change from Category_i to other categories: n_outᵢ=rᵢ−mᵢᵢ and f_outᵢ=(rᵢ−mᵢᵢ)/rᵢ.

In various embodiments, the contingency table may be computed by measuring the number/fraction of incoming samples. In one embodiment, the number/fraction of incoming samples are computed to measure the number/fraction of samples that change to Category_i from all other categories: n_inᵢ=cᵢ−mᵢᵢ and f_inᵢ=(cᵢ−mᵢᵢ)/(N−rᵢ).

In various embodiments, the contingency table may be computed by measuring the number/fraction of additional samples. In one embodiment, the number/fraction of additional samples are computed to measure the overall increase in the number/fraction of samples in Category_i: n_addᵢ=cᵢ−rᵢ and f_addᵢ=(cᵢ−rᵢ)/rᵢ

In one embodiment, the McNemar's Test of Homogeneity of Marginal Distributions may be suggested and applied. Marginal homogeneity occurs when each of the row totals is equal to the corresponding column total, i.e., the number of samples in each category remains the same before and after transition. While originally designed for 2×2 contingency tables, the Generalized McNemar/Stuart-Maxwell Test or the Bhapkar's test can handle higher dimensional tables. In another embodiment, multiple tests may be performed that compare two categories at a time. Depending on the purpose, users may apply the test on all or a subset of the possible category pairs. Comparison may also be made for one category against the rest of the samples pooled together, or between any two groups, with each consisting of a pool of multiple categories. The overall p value may then be given by the minimum of the p values of individual comparisons, with correction for multiple testing using methods such as Bonferroni and false discovery rate (FDR) adjustments. In various embodiments, the McNemar's test statistic (k=2) may be given by

$$Z = \frac{{( {m}_{21} - {m}_{12} )}^{2}}{{m}_{21} + {m}_{12}} ∼ {𝒳}_{1}^{2} .$$

In various embodiments, P values may be computed based on the Chi-square distribution with 1 degree of freedom. In various embodiments, for smaller sample sizes, continuity correction may be automatically applied for improved accuracy.

In various embodiments, the group of edges under study may include a static quantitative trait. In various embodiments, the static quantitative trait may be retrospective, wherein its value is quantitative and remains the same for each sample along a path, e.g., overall survival. For such kind of static quantitative traits, the influence of an edge/path may be evaluated by checking if the sample means change significantly before and after selection by the edge/path. In various embodiments, the goal may be to test for the null hypothesis that samples are randomly selected without replacement from a finite population.

In various embodiments, the quantitative trait follows a normal distribution with mean μ and standard deviation σ in N samples before selection. In this embodiment, n<N samples may be selected by an edge/path and the subset of selected samples may provide a mean of x̅. In various embodiments, the overall impact may be measured by the difference in sample means δ=(x̅−μ) before and after selection. In various embodiments, the finite population correction factor fpc=√((N−n)/(N−1)) may be applied. In such embodiment, the standard deviation of the mean of the selected samples becomes

$${σ}_{\overline{x}} = \frac{σ}{\sqrt{n}} \cdot \sqrt{\frac{N - n}{N - 1}} .$$

The two-sided p value may then be given by

$$p = 1 - sign ( \overline{x} - μ ) \cdot erf ( \frac{\overline{x} - μ}{{σ}_{\overline{x}} \sqrt{2}} ) .$$

In various embodiments, the group of edges under study may include a dynamic quantitative trait. In various embodiments, the dynamic quantitative trait may be longitudinal, wherein its value is quantitative and could change for each sample after traversing an edge, e.g., blood glucose level. For such kind of dynamic quantitative traits, the influence of an edge/path can be evaluated by checking if the value of the quantitative trait tends to increase or decrease in all samples. In various embodiments, the goal may be to test for the null hypothesis that the mean difference in the observed trait values before and after the edge/path in each sample is zero.

In some embodiments, a Dependent T-Test for Paired Samples may be suggested and applied. In one embodiment, the quantitative trait follows a normal distribution. In this embodiment, there are N samples, each with a pair of observed trait values before and after the edge/path under test, wherein X̅_{D} and s_{D} are respectively the average and standard deviation of the pairwise differences between the observed trait values of each sample. In various embodiments, the t-statistic may be given by

$$t = \frac{{\overline{X}}_{D} - {μ}_{0}}{{s}_{D} / \sqrt{N}} ,$$

where μ₀ is the expected mean difference of the trait. While the overall impact may be measured by X̅_{D}, the strength of association may be supported by a p value computed based on the t-distribution with (N−1) degree of freedom:

p=2·Pr(T>|t|),

for two-tailed test,

p=Pr(T>t),

for upper-tailed test (increased trait value for alternative hypothesis) and

p=Pr(T<t),

for lower-tailed test (decreased trait value for alternative hypothesis).

In various embodiments, the graphical structure of the disclosure, formed by a collection of vertices and edges, may be used to effectively represent a sequence of variations across individual genomes of one or multiple cohorts and populations. In various embodiments, a genome-graph may be used to take into account diverse types of genomic variants, including SNVs, indels, haplotypes and structural variants. In some embodiments, the method of graph construction may be used to represent copy number variations (CNVs) by creating CNV graphs and including CNVs with significant effects on the disease as additional elements in the model. In various embodiments, the method of the disclosure may be used to help investigate the influence of mid-to-long range genomic structures in complex regions such as the Major Histocompatibility Complex (MHC), uncover the many weak-to-moderate genetic factors dispersed across the genome for complex disorders and aggregate their influences for disease risk evaluation, and offer a solution for the analysis of whole genome sequencing (WGS) data covering mostly intergenic regions with limited annotations.

In use, the system of the disclosure may be configured to first generate individual treatment pathways for a cohort of patients using user-defined parameters. Exemplary user-defined parameters may include the types and categories of events that qualify for an edge or transition, for example, specific sets of drugs administered to the patient, surgery, a collection of eligible interventions, and the like. Exemplary user-defined parameters additionally include criteria for splitting response states, for example by the immediate response status, such as complete/partial/no response, after a treatment, subtype of a patient based on specific gene signatures, and the like. In some embodiments, the user-defined parameters may include transition graphs purely defined by a sequence of administered treatments, wherein no criteria is needed for splitting of response states. Exemplary user-defined parameters may further include a list of reversible or collapsible events, wherein the order of two or more consecutive events are immaterial and may be collapsed into one combined event for simplifying the pathway. In various embodiments, user-defined parameters may further include a list of additional events which may be collapsed/merged for further simplification of the pathway and graph.

In various embodiments, suitable collapsible events may include similar overlapping edges utilized to increase the aggregate number of samples, hence improving statistical power for detecting associations with phenotypes/outcomes. In various embodiments, edge similarity may be defined by values such as haplotype similarity score for genomic variation graphs or treatment category for state transition graphs. In various embodiments, similar edges may be merged if their effects on the phenotypes/outcomes are in the same direction and the resultant p value or effect measure is stronger than the individual edges. Suitable collapsible events may also include consecutive edges where all samples traversing the second edge completely overlap with one or multiple previous edges and the second edge does not cause any change to the state of any samples. Suitable collapsible events may further include adjacent nodes with the same or highly similar sample states and other connecting edges having insignificant impact on phenotypes/outcomes.

In some embodiments, with the statistical measures computed for individual edges, the overall disease risk of a genome or effectiveness of a clinical pathway towards a favorable treatment outcome may be evaluated by aggregating the statistical evidence of the associated edges. In one embodiment, the number of edges traversed by a genome/pathway significantly associated with a disease/outcome may be counted.

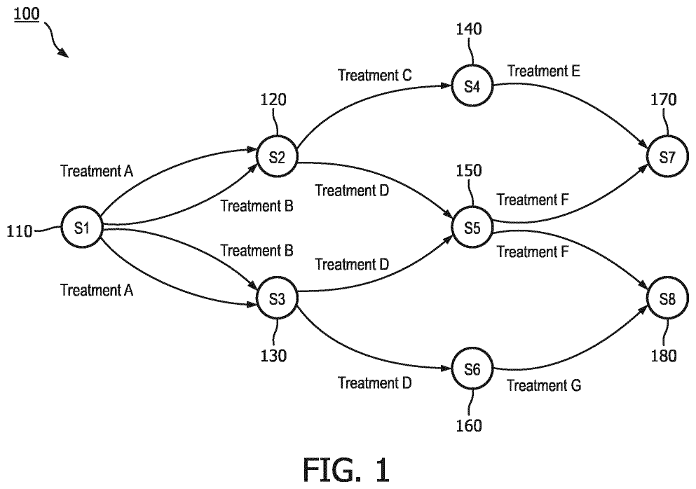

FIG. 1 illustrates a treatment graph 100 that summarizes possible treatment paths and corresponding response state transitions. The treatment graph 100 may first show a patient in a first response state 110, wherein administration of a first treatment A or a second treatment B may be shown to result in a transition to a second response state 120 or a third response state 130. The treatment graph 100 may additionally show the effects of a third treatment C, which, when administered to a patient in a second response state 120 results in a transition to a fourth response state 140. The treatment graph 100 may also show the effects of a fourth treatment D, which, when administered to a patient in a second response state 120 or a third response state 130, results in a transition to a fifth response state 150 or a sixth response state 160. The treatment graph 100 may further show the effects of a fifth treatment E, which, when administered to a patient in a fourth response state 140 results in a transition to a seventh response state 170. The treatment graph 100 may also show the effects of treatment F, which, when administered to a patient in a fifth response state 150, results in a transition to either a seventh response state 170 or an eighth response state 180, as well as the effects of treatment G, which, when administered to a patient in a sixth response state 160 results in a transition to an eighth response state 180.

In various embodiments, the treatment graph 100 may include a series of subgraphs. In various embodiments, the subgraphs may be selected using response state and transition criteria. In various embodiments, the user may confine the graph-based analysis to a subgraph by selecting the regions manually through a user interface that visualizes the graph with support for navigation and user interaction, or by entering a selection criteria. In various embodiments, state transition graphs of the disclosure may be configured to allow the user to select subgraphs that satisfy certain response state/transition criteria at the beginning, in the middle or towards the end of the pathways. In some embodiments, the user may select a subgraph with paths that start with specific types of neoadjuvant chemotherapy followed by surgery, then a complete remission state in the middle and a relapse state at the end.

In various embodiments, different metrics, such as total treatment cost, maximum drug toxicity level, overall severity of side effects, mean and standard deviation of blood pressure and glucose level during the course of treatment, and the like, may be computed for each sample within a selected subgraph based on a user-defined formula. In various embodiments, users may further create additional categorical labels for each sample based on a combination of metrics and criteria of choice. The sample metrics and labels may then be used for downstream analysis.

In various embodiments, the method of the disclosure further allows users to select samples by a defining criteria based on general demographics (e.g., gender and ethnicity), clinical data (e.g., age of diagnosis, smoking status, overall survival, etc.), the computed metrics and labels, or sample IDs. In various embodiments, the method allows for simplification of the subgraph by removing edges not traversed by any selected patient sample.

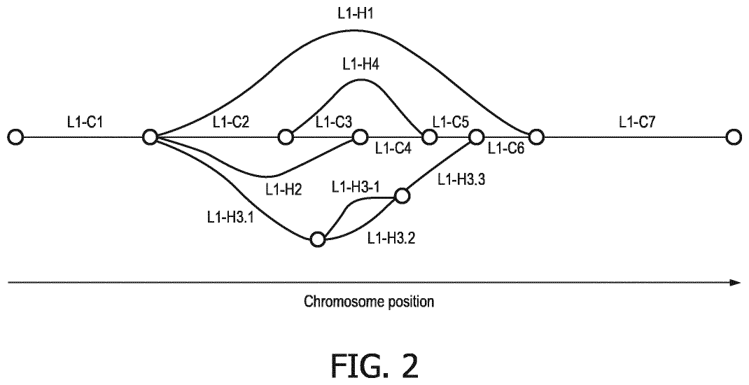

FIG. 2 shows an example of a partial genomic variation graph. The aforementioned techniques of statistical analysis on the influence of an edge, which in this case represents a genomic variation, on the phenotype or disease status can be applied.

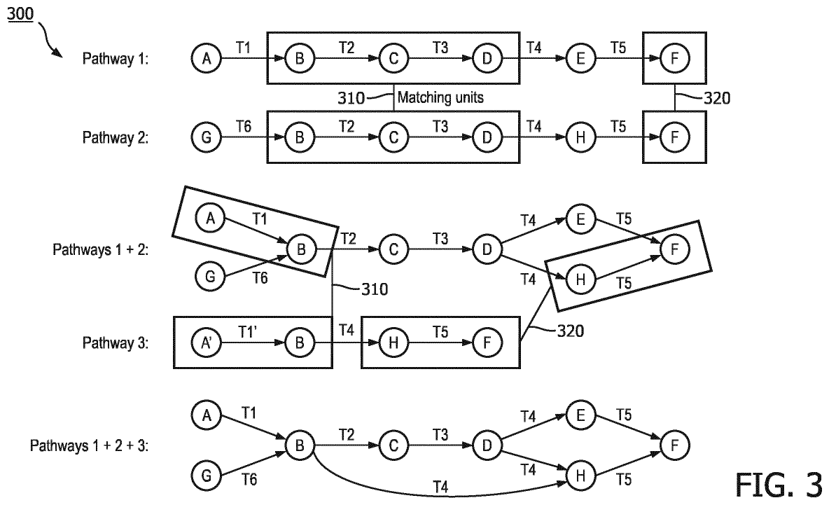

FIG. 3 illustrates an example of the construction of a state transition graph 300 from three sample pathways. As shown in FIG. 3, global states A-H and A′ are represented by circles and Transitions T1-T6 and T1′ are represented by arrows. States A and A′, and Transitions T1 and T1′ may be defined as equivalent by the user. In various embodiments, the graph may be constructed progressively by adding one pathway at a time, with matching units 310, 320 between the graph and the new patient sample identified as anchor points. The resulting state transition graph 300 may also be represented in a table format as shown below, that summarizes an incoming response state, an outgoing response state, a transition event and a traversing pathway for each edge.

| Incoming State | Outgoing State | Transition Event | Pathways |
|---|---|---|---|
| A/A′ | B | T1/T1′ | P1, P3 |
| B | C | T2 | P1, P2 |
| C | D | T3 | P1, P2 |
| D | E | T4 | P1 |
| E | F | T5 | P1 |
| G | B | T6 | P2 |
| D | H | T4 | P2 |
| H | F | T5 | P2, P3 |
| B | H | T4 | P3 |

### EXAMPLE 1

Building the Transition Graph

A state transition graph to evaluate the most and least effective lines of treatments for their existing and future HCC patients is needed at a cancer center. The center desires to build a comprehensive graph of all patients and split the graph into subgraphs according to various stages of disease.

In building the graph, the following events are specified by the clinician as transitions:
i) Trans-arterial chemoembolization (TACE);
ii) TACE with drug-eluting beads (DEB-TACE);
iii) Targeted systemic chemotherapy (sorafenib, sunitinib, linifanib, brivanib, tivantinib, everolimus);
iv) Chemotherapy and TACE combination (sorafenib+TACE);
v) Radioembolization;
vi) Chemotherapy and radioembolization combination (sorafenib+radioembolization);
vii) Percutaneous ethanol injection (PEI);
viii) Cryoablation;
ix) Radiofrequency ablation (RFA);
x) Surgical resection (partial hepatectomy);
xi) Liver transplant.

The clinician supplies the criteria for splitting the response states between each transition. In general, any clinical measurements or intermediate outcomes for therapy guidance, such as Milan criteria (assesses suitability of cirrhosis/HCC patients for liver transplant), drug response and occurrence of metastasis, could be used as criteria for splitting states.

With the transitions and states fully defined, patient treatment and outcome data are retrieved from the center's Electronic Health Records (EHR) and the graph is built to specification by the processor. Since the cancer center would like to evaluate treatment efficacy, association analysis can be performed using one or more of the following classifications or metrics:
i) Complete response;
ii) Objective response;
iii) 5-yr recurrence free survival;
iv) 5-yr overall survival;
v) Mean tumor size.

### EXAMPLE 2

Subgraph Selection for Downstream Analysis

With the state transition graph formed in Example 1, the cancer center would like to evaluate the outcome of patients awaiting liver transplantation with follow-up. Liver transplant (LT) waiting times have increased in recent years causing patients to drop out due to tumor progression so downstaging followed by a minimum observational period is standard practice to keep patients on the waiting list (i.e., Milan criteria must be met). Instead of analyzing the whole graph, criteria should be applied to confine the analysis to a selected subset of patients.

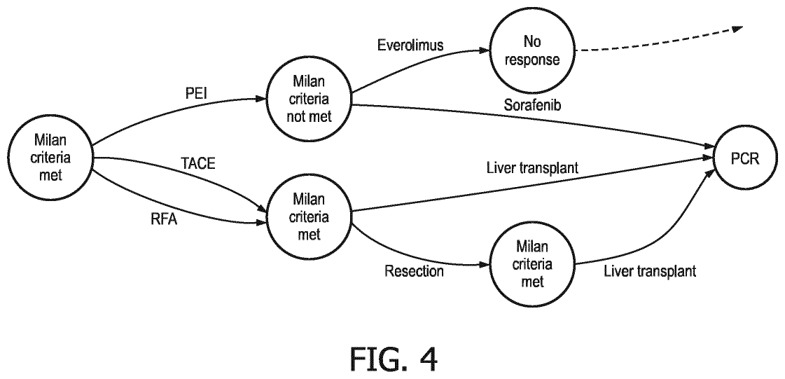

FIG. 4 shows a treatment graph for the HCC patients who have met the Milan Criteria for Liver Transplantation. Three subsets of patients are first treated with PEI, TACE and RFA. Patients treated with TACE and RFA maintain Milan criteria; however, patients treated with PEI experience tumor progression and are no longer eligible. Of those patients who are no longer eligible for LT, one subset is treated with everolimus but experience no response. This subset continues with the next intervention (not shown). Another subset is treated with sorafenib; these patients experience pathologic complete response (PCR). Of those patients who met Milan Criteria with TACE/RFA, one subset found donors and underwent LT, resulting in PCR for the entire subset. The remaining patients who met Milan Criteria with TACE/RFA, time on the waiting list was long enough that resection was administered to keep them eligible. Of these patients, all went on to obtain LT, resulting in PCR.

### EXAMPLE 3

Using Genome Graph for Haplotype Detection

In this example, a clinician seeks to assess whether a patient is at risk for developing type 1 diabetes. While the exact cause of the disease is unknown, certain variants in several human leukocyte antigen (HLA) genes are known to increase the risk of development later in life. Rather than any one variant in particular, certain combinations, or haplotypes, are risk indicators for eventual onset of disease. The HLA region is unique in that it is highly variable even in a healthy population, leading to complex and largely unknown haplotypes.

From a pre-constructed and subsetted (for the HLA region) genomic variation graph, the clinician first selects a subgraph containing a cohort of samples representing patients with and without a confirmed diagnosis of type 1 diabetes, with the goal of identifying the haplotype(s) that most closely match the target patient. Subsequently, the clinician chooses to confine the analysis to the HLA region of chromosome 6, excluding edges in other parts of the genome. The clinician then sets a haplotype similarity threshold of 95% and similar edges are collapsed together with the aim of improving the statistical power of association tests with a larger number of samples per edge. Next, the system calculates adjusted p values for each edge to detect their associations with type 1 diabetes and finds the ones most significant to the analysis.

### EXAMPLE 4

Using Treatment Graphs for Treatment Planning and Outcome Optimization

In this example, a clinician would like to identify the best care plan going forward for a patient with high-risk prostate cancer and would like the care plan optimized for tumor size reduction. In order to apply methods for state transition inference, a statistical framework is first applied to a state transition graph for prostate cancer. Initially, the clinician selects a cohort of patients to populate the high-risk prostate cancer state transition subgraph. This cohort is selected based on a set of attributes shared by the target patient, according to the clinician's own perceived importance and optimization goals: diagnosis, disease stage, demographics, etc. The cohort is not overly restrictive, so as to maintain statistical power as well as to include a diverse set of retrospective clinical pathways and outcomes which are used to produce an optimal model.

Once the subgraph is selected, several starting points are identified in the treatment graph that match the current condition of the target patient. The clinician selects one such starting point (response state), which matches the current state of the target patient, wherein initial treatment must be decided. Subsequent to this state are multiple edges (representing multiple treatments) drawn to multiple outcome states, indicating that some therapies prove more effective than others in the cohort. One such edge, edge A, corresponds with administration of radical external beam radiotherapy; a second edge, edge B, corresponds with administration of androgen deprivation therapy (ADT); a third edge, edge C, corresponds with administration of ADT and subsequent external beam radiotherapy. Subsequent ranking methods are used to inform the clinician of outcomes for patients along each edge; the clinician sees that edge C would likely have the best outcome according to the ranking method chosen and decides to administer ADT and external beam radiotherapy to the patient.

### EXAMPLE 5

Insurance Company Risk Assessment, Therapy Efficacy

An insurance company would like to calculate new premium rates for policy holders. In order to calculate premium rates, and maintain a profit, insurance underwriters want to evaluate the risk that a new policy holder will file a claim against the insurance policy. A life insurance underwriter is calculating premium rates for the policy of a new customer. The underwriter has, among other information, access to the individual's health history, and would like to evaluate the odds of the customer (or customer's family) filing a claim against the policy in the next 30 years. The underwriter also has access to a state transition graph of historical claims and health history data for the insurance company's previous and current customers. The underwriter selects a cohort of customers which match the demographics of the patient. Then, the system splits the paths of customers into two categories; those who filed a claim within 30 years and those who did not. The odds ratio for each category is computed and it is found that the new customer will most likely not file a claim in the next 30 years, and the underwriter subsequently chooses to present the new customer with a less expensive premium rate.

Technical Innovation

There currently exist no known data-driven approaches for determining personalized care pathway management, partly due to lack of data structures to store and associate historical pathway data and also a lack of appropriate analytical methods to make use of it. The graphical structures described herein effectively aggregate and visualize historical patient data parameters to allow for exploration and discovery of trends and associations between treatments and outcomes through downstream analysis. The graphical structures described herein also enable computation of statistical models that make use of the data for aggregated studies, for example, on the effectiveness of certain drugs or treatment courses, or pathway guidance for individual patients optimized for variables such as specific clinical outcomes, cost and minimal side effects.

While one or more features of the embodiments may involve the use of a mathematical formula, the embodiments are in no way restricted solely to a mathematical formula. Nor are they directed to a method of organizing human activity or a mental process. Rather, the complex and specific approach taken by the embodiments, combined with the amount of information processing performed, negate the possibility of the embodiments being performed by human activity or a mental process. Moreover, while a computer or other form of processor may be used to implement one or more features of the embodiments, the embodiments are not solely directed to using a computer as a tool to otherwise perform a process that was previously performed manually.

Nor do these embodiments preempt the general concept of making treatment decisions. Rather, the embodiments disclosed herein take a specific approach (e.g., through event logs, trace sets, clustering algorithms, and weighting and distance measuring models) to solving technological problems that do not preempt, or otherwise restrict the public from practicing the general concept of, allocating healthcare resources.

The methods, processes, and/or operations described herein may be performed by code or instructions to be executed by a computer, processor, controller, or other signal processing device. The code or instructions may be stored in a non-transitory computer-readable medium in accordance with one or more embodiments. Because the algorithms that form the basis of the methods (or operations of the computer, processor, controller, or other signal processing device) are described in detail, the code or instructions for implementing the operations of the method embodiments may transform the computer, processor, controller, or other signal processing device into a special-purpose processor for performing the methods herein.

The modules, stages, models, processors, and other information generating, processing, and calculating features of the embodiments disclosed herein may be implemented in logic which, for example, may include hardware, software, or both. When implemented at least partially in hardware, the modules, models, engines, processors, and other information generating, processing, or calculating features may be, for example, any one of a variety of integrated circuits including but not limited to an application-specific integrated circuit, a field-programmable gate array, a combination of logic gates, a system-on-chip, a microprocessor, or another type of processing or control circuit.

When implemented in at least partially in software, the modules, models, engines, processors, and other information generating, processing, or calculating features may include, for example, a memory or other storage device for storing code or instructions to be executed, for example, by a computer, processor, microprocessor, controller, or other signal processing device. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the methods herein.

It should be apparent from the foregoing description that various exemplary embodiments of the invention may be implemented in hardware. Furthermore, various exemplary embodiments may be implemented as instructions stored on a non-transitory machine-readable storage medium, such as a volatile or non-volatile memory, which may be read and executed by at least one processor to perform the operations described in detail herein. A non-transitory machine-readable storage medium may include any mechanism for storing information in a form readable by a machine, such as a personal or laptop computer, a server, or other computing device. Thus, a non-transitory machine-readable storage medium may include read-only memory (ROM), random-access memory (RAM), magnetic disk storage media, optical storage media, flash-memory devices, and similar storage media and excludes transitory signals.

It should be appreciated by those skilled in the art that any blocks and block diagrams herein represent conceptual views of illustrative circuitry embodying the principles of the invention. Implementation of particular blocks can vary while they can be implemented in the hardware or software domain without limiting the scope of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Accordingly, it is to be understood that the above description is intended to be illustrative and not restrictive. Many embodiments and applications other than the examples provided would be apparent upon reading the above description. The scope should be determined, not with reference to the above description or Abstract below, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

We claim:

1. A computer-implemented method for constructing a state transition graph for treatment, procedure and progression workflows, wherein the method comprises:
obtaining, by one or more computing devices, data that comprises treatment history and clinical data of a cohort of patients;
generating, by the one or more computing devices, individual treatment pathways for individual patients of the cohort of patients using the treatment history and clinical data for the individual patients; wherein the individual treatment pathways are generated using user-defined parameters comprising:
one or more qualifying events;
one or more response states to the one or more qualifying events; and
one or more reversible or collapsible events; and
constructing, by the one or more computing devices, a state transition graph that represents multiple aligned and merged individual treatment pathways comprising the one or more qualifying events, the one or more response states to the one or more qualifying events and the one or more reversible or collapsible events.

2. The method of claim 1, wherein the one or more qualifying events comprises one or more treatment regimens.

3. The method of claim 2, wherein the one or more treatment regimens is selected from the group consisting of a drug regimen, a surgical protocol, a collection of eligible interventions, or combinations thereof.

4. The method of claim 1, wherein the one or more response states is selected from the group consisting of a response status after a treatment; and a subtype of the patient based on a specific gene signature.

5. The method of claim 1, wherein the one or more response states is linked to one or more reports selected from the group consisting of a clinical report, a radiology report, a pathology report, a genomics report, or combinations thereof.

6. The method of claim 1, wherein the constructing comprises adding individual treatment pathways one at a time to the state transition graph.

7. The method of claim 1, wherein the state transition graph comprises edges that correspond to treatments of a similar nature.

8. The method of claim 7, wherein the edges are collapsible.

9. The method of claim 1, wherein the method further comprises constructing one or more subgraphs generated using further user-defined parameters comprising one or more qualifying events; one or more response states to the one or more qualifying events; and one or more reversible or collapsible events.

10. The method of claim 1, further comprising receiving a new individual pathway to add to the state transition graph;
identifying the largest possible matching sequence of state-event-state units between the new individual pathway and the state transition graph as anchor points; and
adding the new individual pathway to the state transition graph, wherein the resulting state transition graph remains acyclic and has the least number of additional response states and edges.

11. A system for processing treatment and clinical data, comprising:
a memory configured to store instructions;
a processor configured to execute the instructions to:
obtain data that comprises treatment history and clinical data of a cohort of patients;
generate individual treatment pathways for individual patients of the cohort of patients using the treatment history and clinical data for the individual patients using user-defined parameters comprising:
one or more qualifying events;
one or more response states to the one or more qualifying events; and
one or more reversible or collapsible events; and
construct a state transition graph that represents multiple aligned and merged individual treatment pathways comprising the one or more qualifying events, the one or more response states to the one or more qualifying events and the one or more reversible or collapsible events.

12. The system of claim 11, wherein the one or more qualifying events comprises one or more treatment regimens.

13. The system of claim 11, wherein the one or more treatment regimens is selected from the group consisting of a drug regimen, a surgical protocol, a collection of eligible interventions, or combinations thereof.

14. The system of claim 101 wherein the one or more response states is selected from the group consisting of a response status after a treatment; and a subtype of the patient based on a specific gene signature.

15. The system of claim 11, wherein the processor is configured to add individual treatment pathways one at a time to the state transition graph.

16. The system of claim 11, wherein the processor is configured to link the one or more response states to one or more reports selected from the group consisting of a clinical report, a radiology report, a pathology report, a genomics report, or combinations thereof.

17. The system of claim 16, wherein the processor is configured to link the one or more response states to one or more genomics reports.

18. The system of claim 11, wherein the processor is configured to construct a state transition graph comprising edges that correspond to treatments of a similar nature.

19. The system of claim 18, wherein the processor is configured to collapse edges corresponding to treatments of a similar nature.

20. The system of claim 11, wherein the processor is further configured to construct one or more subgraphs generated using further user-defined parameters comprising one or more qualifying events; one or more response states to the one or more qualifying events; and one or more reversible or collapsible events.

21. The system of claim 11, where in the processor is further configured to:
receive a new individual pathway to add to the state transition graph;
identify the largest possible matching sequence of state-event-state units between the new individual pathway and the state transition graph as anchor points; and
add the new individual pathway to the state transition graph, wherein the resulting state transition graph remains acyclic and has the least number of additional response states and edges.

22. A non-transitory, machine-readable medium storing instructions for controlling a processor to perform operations which comprise:
obtaining, by one or more computing devices, data that comprises treatment history and clinical data of a cohort of patients;
generating, by the one or more computing devices, individual treatment pathways for individual patients of the cohort of patients using the treatment history and clinical data for the individual patients; wherein the individual treatment pathways are generated using user-defined parameters comprising:
one or more qualifying events;
one or more response states to the one or more qualifying events; and
one or more reversible or collapsible events; and
constructing, by the one or more computing devices, a state transition graph that represents multiple aligned and merged individual treatment pathways comprising the one or more qualifying events, the one or more response states to the one or more qualifying events and the one or more reversible or collapsible events.
